(19) United States
(12) Patent Application Publication
(10) Pub. No.: US 2020/0158678 A1
(43) Pub. Date: May 21, 2020

(54) NANOSTRUCTURED GRAPHENE-MODIFIED GRAPHITE PENCIL ELECTRODE SYSTEM FOR SIMULTANEOUS DETECTION OF ANALYTES

(71) Applicant: King Fahd University of Petroleum and Minerals, Dhahran (SA)

(72) Inventors: Abdel-Nasser M. Kawde, Dhahran (SA); Nadeem Baig, Dhahran (SA)

(73) Assignee: King Fahd University of Petroleum and Minerals, Dhahran (SA)

(21) Appl. No.: 16/196,232

(22) Filed: Nov. 20, 2018

(51) Int. Cl. G01N 27/30; G01N 27/48; G01N 27/403; G01N 33/487

(52) U.S. Cl. CPC G01N 27/308; G01N 33/48707; G01N 27/403; G01N 27/48

(57) ABSTRACT

A graphene-modified graphite pencil electrode (GPE) system and a method for simultaneous detection of multiple anylates such as dopamine, uric acid, and L-tyrosine in a solution. The electrode system includes a graphene-modified graphite pencil working electrode comprising a graphite pencil base electrode and a layer of three dimensional nanostructured multiwall network forming concave shape structures on the surface of the graphite pencil base electrode, a counter electrode, and a reference electrode. The method comprises contacting the solution with the graphene-modified GPE system and conducting voltammetry, preferably square wave voltammetry, to detect the L-tyrosine concentration in the solution.

### BACKGROUND OF THE INVENTION

### Technical Field

The present disclosure relates to graphite pencil electrode (GPE) modified with a network of nanostructured vertical walls having concave-shaped three dimensional (3D) methylene blue (MTLB-GR)-graphene (GR) structures on the surface. The electrode may be integrated into electrochemical system for use in a method for the simultaneous detection of analytes.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, is neither expressly nor impliedly admitted as prior art against the present invention.

Sensing of small biomolecules is an invaluable tool for diagnosing diseases and evaluating the health condition of a subject [Abellán-Llobregat et al. “Portable electrochemical sensor based on 4-aminobenzoic acid-functionalized herringbone carbon nanotubes for the determination of ascorbic acid and uric acid in human fluids” Biosens. Bioelectron. 109 (2018) 123-131, doi:10.1016/J.BIOS.2018.02.047], as well as monitoring treatment efficacy. Dopamine, uric acid, and L-tyrosine are found in biological fluids and are useful biomarkers for several diseases. Dopamine is a neurotransmitter and hormone contributing to feelings, learning, mood, attention, and behavior of humans [Beitollahi et al. “A Novel Strategy for Simultaneous Determination of Dopamine and Uric Acid Using a Carbon Paste Electrode Modified with CdTe Quantum Dots” Electroanalysis. 27 (2015) 524-533, doi:10.1002/elan.201400635.]. The role of dopamine is evident in renal, nervous and cardiovascular systems [Huang et al. “A high performance electrochemical biosensor based on Cu₂O-carbon dots for selective and sensitive determination of dopamine in human serum” RSC Adv. 5 (2015) 54102-54108. doi:10.1039/C5RA05433H]. Abnormal levels of dopamine are associated with several diseases such as Parkinson's disease, schizophrenia, and Huntington [Yildirim et al. “Turn-on Fluorescent Dopamine Sensing Based on in Situ Formation of Visible Light Emitting Polydopamine Nanoparticles, Anal. Chem. 86 (2014) 5508-5512. doi:10.1021/ac500771q]. Uric acid is the oxidation product of purine metabolism [Xie et al. “Facile ultrasonic synthesis of graphene/SnO₂ nanocomposite and its application to the simultaneous electrochemical determination of dopamine, ascorbic acid, and uric acid” J. Electroanal. Chem. 749 (2015)]. Elevated levels of uric acid in biological fluid is diagnostic for several diseases including hyperuricemia, gout, and Lesch-Nyhan syndrome [Özcan et al. “Preparation of poly(3,4-ethylenedioxythiophene) nanofibers modified pencil graphite electrode and investigation of over-oxidation conditions for the selective and sensitive determination of uric acid in body fluids” Anal. Chim. Acta. 891 (2015) 312-320, doi:10.1016/j.aca.2015.08.015; Zhang et al. “Carbon nanohorns/poly(glycine) modified glassy carbon electrode: Preparation, characterization and simultaneous electrochemical determination of uric acid, dopamine and ascorbic acid” J. Electroanal. Chem. 760 (2016) 24-31. doi:10.1016/j.jelechem.2015.11.035, and Baig et al. “A cost-effective disposable graphene-modified electrode decorated with alternating layers of Au NPs for the simultaneous detection of dopamine and uric acid in human urine” RSC Adv. 6 (2016) 80756-80765. doi:10.1039/C6RA10055D.]. L-tyrosine is an essential amino acid and a precursor of dopamine, epinephrine, and norepinephrine. Also, it has a regulatory function of pituitary, thyroid, and adrenal glands [D'Souza et al. “A multi-walled carbon nanotube/poly-2,6-dichlorophenolindophenol film modified carbon paste electrode for the amperometric determination of L-tyrosine” RSC Adv. 5 (2015) 91472-91481. doi:10.1039/C5RA18329D]. Increased level of L-tyrosine is diagnostic of tyrosinemia, whereas decreased level of L-tyrosine is associated with alkaptonuria and albinism. The detection of L-tyrosine in urine is a biomarker for certain cancers, and the concentration of L-tyrosine that is 50% higher than normal in urine is associated with high probability for the presence of tumor in a patent [Gu et al. “A facile sensitive L-tyrosine electrochemical sensor based on a coupled CuO/Cu₂O nanoparticles and multi-walled carbon nanotubes nanocomposite film” Anal. Methods. 7 (2015) 1313-1320. doi:10.1039/C4AY01925C]. Among the different analytical tools available, electrochemical sensors are the preferred choice to design and fabricate sensitive and selective instruments to detect small molecules such as uric acid, dopamine, and L-tyrosine [Hussain et al. “Development of selective Co²⁺ ionic sensor based on various derivatives of benzenesulfonohydrazide (BSH) compound: An electrochemical approach” Chem. Eng. J. 339 (2018) 133-143, doi:10.1016/j.cej.2018.01.130].

After isolation in 2004, graphene has been considered for many uses in various fields. It has a one atom thick two dimensional (2D) honeycomb-like structure formed by sp² carbon atoms [Song et al. “Biosensors and Bioelectronics Recent advances in electrochemical biosensors based on graphene two-dimensional nanomaterials” Biosens. Bioelectron. 76 (2016) 195-212. doi:10.1016/j.bios.2015.07.002]. Among 2D materials, the desired graphene properties make graphene the material of choice in many fields for various applications. It is under active investigation for use in transparent conductors, solar cells, batteries, fuel cells, field emission display, and electrochemical sensors [Liu et al. “Biological and chemical sensors based on graphene materials” Chem. Soc. Rev. 41 (2012) 2283-2307, doi:10.1039/C1CS15270J; and Shao et al. “Graphene Based Electrochemical Sensors and Biosensors: A Review, Electroanalysis” 22 (2010) 1027-1036. doi:10.1002/elan.200900571] It has gained considerable attention in the field of electrochemical sensing due to rapid charge transfer, low resistance, and wide potential window [Wu, Q. He, C. Tan, Y. Wang, H. Zhang, Graphene-Based Electrochemical Sensors, Small. 9 (2013) 1160-1172. doi:10.1002/smll.201202896.]. Graphene is used in the development of numerous sensitive sensors for detecting various analytes. The sensitivity of the sensors can be further improved using graphene in combination with other nanomaterials. The metal-graphene, metal oxide-graphene, and polymer-based graphene nanocomposite are used in the development of sensors, optical devices, and catalysts [Cao et al. “In situ Controllable Growth of Prussian Blue Nanocubes on Reduced Graphene Oxide: Facile Synthesis and Their Application as Enhanced Nanoelectrocatalyst for H₂O₂ Reduction” ACS Appl. Mater. Interfaces. 2 (2010) 2339-2346. doi:10.1021/am100372m.]. Also, polymethylene blue graphene composite (PMB-GR) displays interesting behavior on the surface of various electrode systems. The PMB-GR/carbon ionic liquid electrode was used as a detector for dopamine with high sensitivity [Sun et al. “Poly(methylene blue) functionalized graphene modified carbon ionic liquid electrode for the electrochemical detection of dopamine, Anal. Chim. Acta. 751 (2012) 59-65. doi:10.1016/j.aca.2012. 09.006]. Similarly, dopamine grafted graphene oxide/poly(methylene blue) on glassy carbon electrode surface was used for dopamine sensing [Gorle et al. “Electrochemical sensing of dopamine at the surface of a dopamine grafted graphene oxide/poly(methylene blue) composite modified electrode” RSC Adv. 6 (2016) 19982-19991. doi:10.1039/C5RA25541D]. Also, NADH was determined using Graphene/Poly(methylene blue)/AgNPs Composite on Paper [Topçu et al. “Free-standing Graphene/Poly(methylene blue)/AgNPs Composite Paper for Electrochemical Sensing of NADH” Electroanalysis. 28 (2016) 2058-2069. doi:10.1002/elan.201600108.] and Graphene/Methylene Blue Nanocomposite thin films on Au electrode [Erçarlkcl et al. “Fatigue properties of highly oriented polypropylene tapes and all-polypropylene composites” Polym. Polym. Compos. (2016). doi:10.1002/pc].

The intrinsic characteristics of 2D graphene are compromised by stacking of the graphene into graphitic form. Recently, a new three dimensional (3D) architecture of graphene is introduced to overcome the stacking issue [Baig et al. “Electrodes modified with 3D graphene composites: a review on methods for preparation, properties and sensing applications, Microchim” Acta. 185 (2018) 283. doi:10.1007/s00604-018-2809-3.]. The 3D architecture provided more active surface area for the electrochemical reaction and can be obtained by several methods including hydrothermal, chemical vapor deposition, lithography, and electrochemical methods. Also, the separation of graphene layers has been achieved by introduction of spacers such as carbon nanotubes, carbon nanofibers, metals nanoparticles, and conductive polymers [Zhang et al. “Self-assembly synthesis of a hierarchical structure using hollow nitrogen-doped carbon spheres as spacers to separate the reduced graphene oxide for simultaneous electrochemical determination of ascorbic acid, dopamine and uric acid, Anal. Methods. 5 (2013) 3591, doi:10.1039/c3ay40572a; Li et al. “Fabrication of High-Surface-Area Graphene/Polyaniline Nanocomposites and Their Application in Supercapacitors” ACS Appl. Mater. Interfaces. 5 (2013) 2685-2691, doi:10.1021/am4001634; Cui et al. “Electrochemical sensor for epinephrine based on a glassy carbon electrode modified with graphene/gold nanocomposites” J. Electroanal. Chem. 669 (2012) 35-41, doi:10.1016/j.jelechem.2012.01.021; Liu et al. “Palladium Nanoparticles Embedded into Graphene Nanosheets: Preparation, Characterization, and Nonenzymatic Electrochemical Detection of H₂O₂” Electroanalysis. 26 (2014) 556-564, doi:10.1002/elan.201300428; Rakhi et al. “High performance supercapacitors using metal oxide anchored graphene nanosheet electrodes” J. Mater. Chem. 21 (2011) 16197, doi:10.1039/cljm12963e; Li et al. “Flexible Solid-State Supercapacitor Based on Graphene-based Hybrid Films” Adv. Funct. Mater. 24 (2014) 7495-7502, doi:10.1002/adfm.201402442; and Fu et al. “Facile one-pot synthesis of graphene-porous carbon nanofibers hybrid support for Pt nanoparticles with high activity towards oxygen reduction” Electrochim. Acta. 215 (2016) 427-434, doi:10.1016/j.electacta.2016.08.111]. The 3D architecture of graphene provides increased sensitivity to an electrode which enhances the effectiveness of the electrode in sensing analytes.

Sensors are the primary recognition method for direct and indirect monitoring of various biomarkers. Sensitivity, selectivity, and cost are the primary challenges in developing disposable sensors. In the past few years, the commonly used graphite pencil was developed as a working electrode for electrochemical detection of analytes [Özcan et al. “Preparation of a disposable and low-cost electrochemical sensor for propham detection based on over-oxidized poly(thiophene) modified pencil graphite electrode” Talanta. 187 (2018) 125-132, doi:10.1016/j.talanta.2018.05.018]. The surface of the pencil electrode is activated and modified with a nano-material for detecting many analytes [Baig et al. “A cost-effective disposable graphene-modified electrode decorated with alternating layers of Au NPs for the simultaneous detection of dopamine and uric acid in human urine” RSC Adv. 6 (2016) 80756-80765, doi:10.1039/C6RA10055D; Kawde et al. “Graphite pencil electrodes as electrochemical sensors for environmental analysis: a review of features, developments, and applications” RSC Adv. 6 (2016) 91325-91340, doi:10.1039/C6RA17466C; Kawde et al. “A facile fabrication of platinum nanoparticle-modified graphite pencil electrode for highly sensitive detection of hydrogen peroxide” J. Electroanal. Chem. 740 (2015) 68-74, doi:10.1016/j.jelechem.2015.01.005; and Baig et al. “A novel, fast and cost effective graphene-modified graphite pencil electrode for trace quantification of <scp>1</scp>-tyrosine” Anal. Methods. 7 (2015) 9535-9541. doi:10.1039/C5AY01753J]. It is abundantly available at low cost and can be modified with relative ease at a controllable exposed surface area. Various methods are being explored to enhance its sensitivity and make it a valuable tool in electrochemical sensing.

It is therefore one object of the present disclosure to provide a graphite pencil electrode modified with methylene blue and having a surface structure with a nanostructured network of vertical walls forming concave shaped 3D MTLB/GR structures. The electrode has high sensitivity and selectivity in detecting and quantifying dopamine, uric acid, and L-tyrosine simultaneously and can be obtained at a low cost.

### BRIEF SUMMARY OF THE INVENTION

According to a first aspect, the present disclosure relates to a graphene-modified graphite pencil electrode system. The electrode system includes a graphene-modified graphite pencil working electrode comprising a graphite pencil base electrode modified with three-dimensional architecture of vertical walls network of methylene blue (MTLB)/graphene (GR) composite forming concave structures on the surface of the graphite pencil base electrode, a counter electrode, and a reference electrode

In a preferred embodiment, the MTLB/GR-modified pencil working electrode has an electro active surface areas determined for dopamine, uric acid, and L-tyrosine of about 2.35 cm², 1.43 cm², and 0.30 cm², respectively.

In another preferred embodiment, the MTLB/GR-modified pencil working electrode is obtained by electrochemical reduction of a composition comprising MTLB and graphene oxide (GO) at the surface of a graphite pencil electrode by scanning from about −1.4 to 0.5 V at scan rate in the range of 0.02 to 0.04 V/s for 4 to 6 cycles.

In a more preferred embodiment, the composition comprising MTLB at a concentration in the range of 0.4 to 0.6 mM and GO at a concentration of at least 2 mg/mL.

In another preferred embodiment, the charge transfer resistance of the graphene-modified graphite pencil working electrode is at least 95% less than the charge transfer resistance of the graphite pencil base electrode as the working electrode, and wherein the electroactive area of the graphene-modified graphite pencil working electrode is at least 5 times as that of the graphite pencil base electrode as the working electrode.

A second aspect of the invention is directed to a method of modifying graphite pencil electrode comprising:
disolving MTLB in water at a concentration in the range of 0.4 to 0.6 mM to form an MTLB solution,
suspending GO in the solution in an amount in the range of 1.5 to 3.0 mg/mL, and
reducing MTLB-GO on the pencil electrode surface by sweeping electrode potential from about −1.4 to about 0.5 V over 4 to 7 cycles at scanning rate in the range of 0.02 to 0.04 V/s

In a preferred embodiment of the method the MTLB concentration is 0.5 mM.

In another preferred embodiment of the method, the amount of GO is 2 mg/mL.

In another preferred embodiment of the method, the sweeping electrode potential −1.4 to 0.5 V over 5 cycles at scanning rate of 0.03 V/s.

A third aspect of the invention is directed to a method of detecting dopamine, uric acid, L-tyrosine, or combination thereof simultaneously in a solution, comprising:
contacting the solution with the graphene-modified graphite pencil electrode system of the invention, and
conducting square wave voltammetry to detect one or more concentration of dopamine, uric acid, and L-tyrosine in the solution, wherein the conducting square wave voltammetry comprises:
(a) applying a pulsed potential to the MTLB/GR-modified graphite pencil working electrode while sweeping the potential of the MTLB/GR-modified graphite pencil working electrode from a potential that is less than an oxidation peak potential of dopamine, uric acid, and L-tyrosine in the solution and defined as the adsorption potential positively to a potential that is at least the oxidation peak potential of dopamine, uric acid, and L-tyrosine in the solution, and
(b) recording the amount of a forward pulse current and a reverse pulse current during each square wave cycle.

In a preferred embodiment of the method, the amplitude of the pulsed potential is in the range 10 to 100 mV.

In another preferred embodiment of the method, the voltage step of the square wave voltammetry is in the range of 2 to 10 mV.

In another preferred embodiment of the method, the pH of the solution ranges from about 5 to 7.
In another preferred embodiment of the method, the frequency of the pulsed potential is in the range of about 25 to 75 Hz.

In another preferred embodiment of the method, the oxidation peak potential of dopamine in the range of 0.10 to 0.20 V, uric acid in the range 0.25 to 0.35 V, and L-tyrosine in the range of 0.5 V to 0.7 V in the solution.

In another preferred embodiment of the method, the sweeping potential of the MTLB/GR-modified graphite pencil working electrode from the adsorption potential is to adsorb dopamine, uric acid, and L-tyrosine in the solution to the surface of the MTLB/GR-modified graphite pencil working electrode.

In another preferred embodiment of the method, the adsorption time is in the range of 100 to 200 seconds.

In another preferred embodiment of the method, the lowest detectable limit of dopamine, uric acid, and L-tyrosine concentrations in the solution are about 15, 27, and 247 nM, respectively.

In another preferred embodiment of the method, the solution further comprises one or more selected from the group consisting of ascorbic acid, L-phenylalanine, L-alanine, glucose, fructose, L-methionine, uric acid, ascorbic acid, Na⁺, K⁺, Li⁺, Ni²⁺, SO₄²⁻, and Cl⁻.

In another preferred embodiment of the method, the solution comprises at least one selected from the group consisting of whole blood, plasma, serum, saliva, sweat, urine, washes of tissues, extracts of tissues, amniotic fluid, placental fluid, a pharmaceutical composition, and a dietary composition.

In another preferred embodiment, the method further comprising plotting the difference in current between the forward pulse current and the reverse pulse current during each square wave cycle, the difference in current represented by I, against the applied potential of the graphene-modified graphite pencil working electrode, the applied potential represented by E, to obtain a square wave voltammogram, and measuring the magnitudes of peak changes in I in the square wave voltammogram.

In another preferred embodiment of the method, the magnitude of the peak change in I occurring at the dopamine, uric acid, and L-tyrosine oxidation peaks potential in the square wave voltammogram linearly correlates with the concentrations of dopamine and uric acid in the range of 50 to 1000 nM, and L-tyrosine in the range from about 0.7 μM to 30 μM in the solution.

In a more preferred embodiment, the method:
contacting the solution with the graphene-modified graphite pencil electrode system of the invention, and
conducting square wave voltammetry to determine dopamine, uric acid, and L-tyrosine concentration in the solution, wherein the conducting square wave voltammetry comprises:
(a) applying a pulsed potential to the graphene-modified graphite pencil working electrode while sweeping the potential of the graphene-modified graphite pencil working electrode from a potential that is less than an oxidation peak potential of uric acid in the solution and defined as the adsorption potential positively to a potential that is at least the oxidation peak potential of L-tyrosine in the solution, and
(b) recording the amount of a forward pulse current and a reverse pulse current during each square wave cycle,
wherein the square wave voltammetry includes conditions in which: the frequency is in the range of 40 to 60 Hz; the amplitude is is in the range of 20 to 80 mV; the voltage step is 2-10 mV; the adsorption potential is 0.0-0.4 V; the adsorption time is in the range 100 to 200 seconds; and the pH value is in the range of 6.0 to 8.0.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows oxidation peak current response of cyclic voltammetry (CV) for 0.5 mM in 0.1 mM PBS (a) L-tyrosine (b) dopamine, and (c) uric acid at various concentration of MTLB containing 3 mg/mL GO.

FIG. 2 shows oxidation peak current response of CVs for 0.5 mM in 0.1 mM PBS (a) L-tyrosine (b) dopamine, and (c) uric acid at various concentrations of GO containing 0.5 mM MTLB.

FIG. 3 shows oxidation peak current response of CVs for 0.5 mM in 0.1 mM PBS (a) L-tyrosine (b) dopamine and (c) uric acid at a various reaction time of MTLB-GO composite.

FIG. 4 shows oxidation peak current response of CVs for 0.5 mM in 0.1 mM PBS (a) L-tyrosine (b) dopamine and (c) uric acid at various scan rates (ν=mV/s) for reduction of MTLB-GO on GPE surface.

FIG. 5 shows the scan window for reduction of MTLB+GO composite for 0.5 mM (a) dopamine (b) uric acid, and (c) L-tyrosine in 0.1 mM PBS.

FIG. 6 shows the reduction cycles for MTLB-GO composite on GPE surface for 0.5 mM (a) L-tyrosine (b) dopamine, and (c) uric acid in 0.1 mM PBS.

FIG. 7 shows the effect of different sensing medium on 0.5 mM dopamine, uric acid, and L-tyrosine.

FIG. 8A shows FE-SEM image at 500 nm magnification of bare/GPE surface.

FIG. 8B shows FE-SEM image at 500 nm magnification of MTLB/GPE surface.

FIG. 8C shows FE-SEM image at 500 nm magnification of GR/GPE surface.

FIG. 8D shows FE-SEM image at 500 nm magnification of 3D-MTLB-GR/GPE surface.

FIG. 9 shows Raman spectra of (a) bare GPE (b) GR/GPE, and (c) 3D-MTLB-GR/GPE.

FIG. 10A shows cyclic voltammogram was acquired using GR/GPE from 0.1 M PBS solution comprising 0.2 mM dopamine at scan rates of (a) 0.05, (b) 0.1, (c) 0.15, (d) 0.2, and (e) 0.25ν. The inset shows the linear relationship between current and the square root of the scan rates.

FIG. 10B shows cyclic voltammogram was acquired using 3D-MTLB-GR/GPE from 0.1 M PBS solution comprising 0.2 mM dopamine at scan rates of (a) 0.05, (b) 0.1, (c) 0.15, (d) 0.2, and (e) 0.25ν. The inset shows the linear relationship between current and the square root of the scan rates.

FIG. 10C shows cyclic voltammogram was acquired using GR/GPE from 0.1 M PBS solution comprising 0.2 mM uric acid at scan rates of (a) 0.05, (b) 0.1, (c) 0.15, (d) 0.2, and (e) 0.25ν. The inset shows the linear relationship between current and the square root of the scan rates.

FIG. 10D shows cyclic voltammogram was acquired using 3D-MTLB-GR/GPE from 0.1 M PBS solution comprising 0.2 mM uric acid at scan rates of (a) 0.05, (b) 0.1, (c) 0.15, (d) 0.2, and (e) 0.25ν. The inset shows the linear relationship between current and the square root of the scan rates.

FIG. 10E shows cyclic voltammogram acquired using GR/GPE from 0.1 M PBS solution comprising 0.5 mM L-tyrosine at scan rates of (a) 0.01 (b) 0.02, (c) 0.04, (d) 0.05, (e) 0.08, and (f) 0.1ν. The inset shows the linear relationship between current and the square root of the scan rates.

FIG. 10F shows cyclic voltammogram acquired using 3D-MTLB-GR/GPE from 0.1 M PBS solution comprising 0.5 mM L-tyrosine at scan rates of (a) 0.01 (b) 0.02, (c) 0.04, (d) 0.05, (e) 0.08, and (f) 0.1ν. The inset shows the linear relationship between current and the square root of the scan rates.

FIG. 11A shows Nyquist plot of 5 mM K₃Fe(CN)₆/K₄Fe(CN)₆ in 0.1 M KCl solution on (a) bare-GPE, and (b) 3D-MTLB-GR/GPE (c) GR/GPE, and (d) MTLB/GPE upon application of frequency range from 0.01 Hz to 100 kHz.

FIG. 11B shows CVs of (a) bare GPE, (b) MTLB/GPE, (c) GR/GPE, (d) MTLB-GR/GPE were recorded at 0.1 V/s in 0.1M PBS solution containing 0.2 mM dopamine, uric acid and 0.5 mM L-tyrosine.

FIG. 12A shows cyclic voltammograms in 0.1 M PBS solution containing 0.4 mM (Aa) L-tyrosine, 0.2 mM (Ab) uric acid and (Ac) dopamine at various pH values from 5 to 7.0 at 3D-MTLB-GR/GPE.

FIG. 12B is a plot of peak current vs pH of (a) L-tyrosine, (b) uric acid, and (cA) dopamine. Inset is showing the relationship between the peak potential and pH of the sensing medium.

FIG. 13 shows the voltammetric technique for dopamine, uric acid and L-tyrosine using 3D-MTLB-GR/GPE in 0.1 mM PBS.

FIG. 14A is a plot of the oxidation peak current vs. amplitude scanned for 20 μM dopamine and uric acid, and 40 μM L-tyrosine.

FIG. 14B is a plot of the oxidation peak current vs. frequency scanned for 10 μM dopamine and uric acid, and 20 μM L-tyrosine.

FIG. 14C is a plot of the oxidation peak current vs. adsorption time for 5 μM dopamine and uric acid, and 40 μM L-tyrosine obtained in 0.1 M PBS buffer, pH 6.0, using square wave voltammetry.

FIG. 15A shows square wave voltammograms of dopamine and uric acid at various concentrations: (a) 50 nM, (b) 100 nM, (c) 2000 nM, (d) 4000 nM, (e) 6000 nM, (f) 0 8000 nM, (g) 10000 nM, and L-tyrosine (a′) 0.7 μM, (b′) 0.9 μM, (c′) 10 μM, (d′) 15 μM, (e′) 20 μM, (f′) 25 μM, (g′) 30 μM.

FIG. 15B shows the linear relationships between I (μA) and the concentrations of (a) dopamine, (b) uric acid and (c) L-tyrosine.

FIG. 15C shows square wave voltammograms of dopamine at various concentrations: (a) 2 μM, (b) 4 μM, (c) 6 μM, and (d) 8 μM in the presence of both 4 μM uric acid and 20 μM L-tyrosine. The inset shows the linear relationship between I (μA) and the concentrations (μM).

FIG. 15D shows square wave voltammograms of uric acid at various concentrations: (a) 2 μM, (b) 4 μM, (c) 6 μM, (d) 8 μM, (e) 10 μM in the presence of 2 μM dopamine and 20 μM L-tyrosine. The inset shows the linear relationship between I (μA) and the concentrations (μM).

FIG. 15E shows square wave voltammograms of L-tyrosine concentrations: (a) 10 μM, (b) 15 μM, (c) 20 μM, (d) 25 μM in the presence of 2 μM dopamine and uric acid. The inset shows the linear relationship between I (μA) and the concentrations (μM).

### DETAILED DESCRIPTION OF THE EMBODIMENTS

All publications mentioned herein are incorporated herein by reference in full for the purpose of describing and disclosing the methodologies, which are described in the publications, which might be used in connection with the description herein. Nothing herein is to be construed as an admission that the inventors are not entitled to antedate such disclosure by virtue of prior disclosure. Also, the use of “or” means “and/or” unless stated otherwise. Similarly, “comprise,” “comprises,” “comprising” “include,” “includes,” and “including” are interchangeable and not intended to be limiting.

As used herein, the term “about” refers to an approximate number within 20% of a stated value, preferably within 15% of a stated value, more preferably within 10% of a stated value, and most preferably within 5% of a stated value. For example, if a stated value is about 8.0, the value may vary in the range of 8±1.6, ±1.0, ±0.8, ±0.5, ±0.4, ±0.3, ±0.2, or ±0.1.

Disclosed herein are a MTBL/GR-modified graphite pencil electrode (GPE) and corresponding system, and methods of using the electrode and system to detect dopamine, uric acid, and L-tyrosine simultaneously, especially at a very low concentration, in a solution. The first aspect of the invention is directed to an electrode system that includes an MTBL/graphene-modified graphite pencil working electrode comprising a graphite pencil base electrode modified with an MTLB/GR composite in the form of a three-dimensional architecture having a network of vertical walls forming concave structures on the surface of the graphite pencil base electrode, a counter electrode, and a reference electrode.

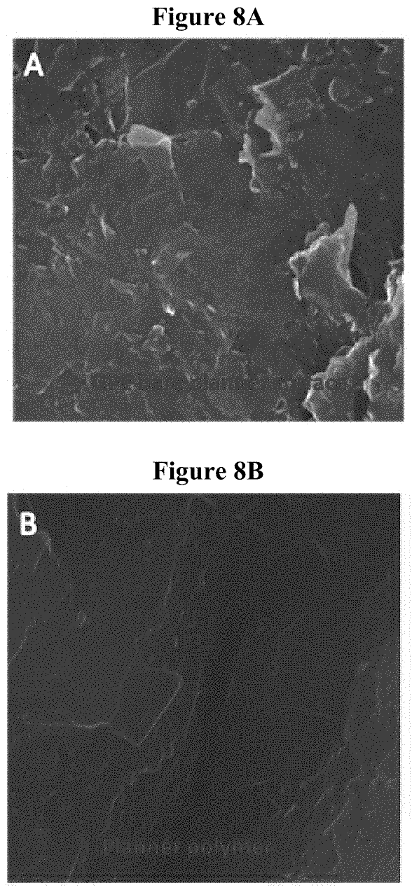

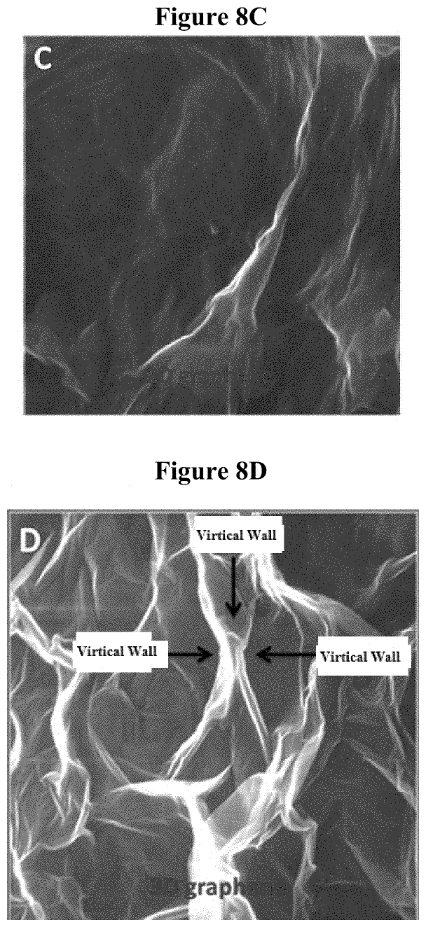

The concave structures are defined by a network of walls, extensions and/or protuberances formed on the graphite surface. FIG. 8D shows Fe-SEM an image of MTLB/GR composite having concave structures formed by a network of vertical and intersecting walls. The walls may be straight, curved, angular, branched, continuous, or fragmented. The overall shape of the concave structures can be any regular or irregular geometrical shape, such as but not limited to square, rectangular, circular, rectangular, and the like. The heights of vertical walls from the surface of the graphite are varied in the range of 0.5 nm to 100 nm, preferably in the range of 5 nm to 90 nm, more preferably in the range of 10 nm to 60 nm, and most preferably in the range of 25 nm to 50 nm. Also, the observed width of the walls may vary. Walls width may be at least 0.5 nm, 1.0 nm, 2.0 nm, 3 nm, 5 nm, 10 nm, 15 nm, 20 nm, 30 nm, 40 nm, and/or 50 nm.

The three dimensional MTLB/GR composite covers at least 70%, preferably at least 80%, more preferably at least 90%, or more preferably at least 95% of the surface of graphite pencil electrode.

The MTLB/GR-modified graphite pencil working electrode with MTLB/GR composite of the invention displays major improvements in electrochemical characteristics compared to an unmodified graphite pencil base electrode.

The charge transfer resistance of the MTLB/GR-modified graphite pencil working electrode of the invention can be determined from the Nyquist plot obtained by electrochemical impedance spectroscopy. In some embodiments, the charge transfer resistance of the MTLB/GR-modified graphite pencil working electrode is at least 70%, preferably at least 80%, preferably at least 90%, or more preferably at least 95% less than that of the graphite pencil base electrode without MTLB/GR modification.

In some embodiments, the electron transfer rate constant of the MTLB/GR-modified graphite pencil working electrode is at least 7 times, preferably at least 10 times, more preferably at least 15 times, more preferably at least 18 times, more preferably at least 25 times, or more preferably at least 30 times as that of the graphite pencil base electrode without MTLB/GR modification.

In some embodiments, the electroactive area of the MTLB/GR-modified graphite pencil working electrode is at least 3 times, preferably at least 5 times, more preferably at least 7 times, or more preferably at least 10 times as that of the graphite pencil base electrode without MTLB/GR modification.

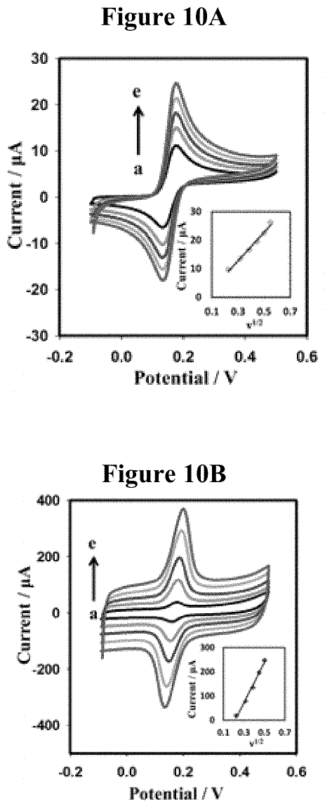

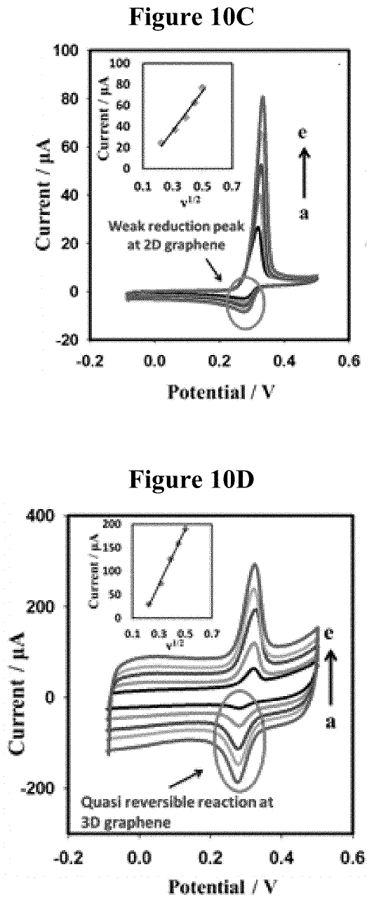

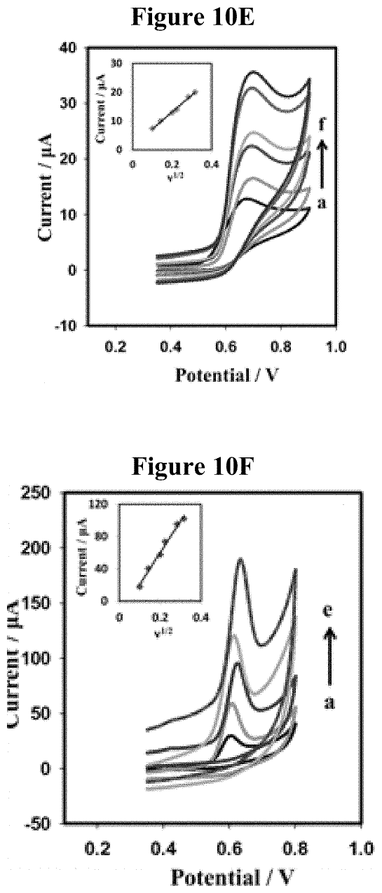

Measured for a solution of 0.2 mM of dopamine and uric acid in 0.1 M PBS, and tyrosine 0.5 mM in 0.1 PBS, the unmodified graphite electrode displays an electroactive surface area of 0.141, 0.453, and 0.045 cm², respectively (see FIGS. 10A-10C′). The electroactive surface of the MTLB/GR modified graphite pencil electrode substantially increased compared to the unmodified graphite pencil electrode for dopamine, uric acid, and L-tyrosine. In one embodiment, the modified electroactive surface area of the modified electrode as measured with dopamine is in the range of 1.0 to 3.0 cm², preferably in the range of 1.8 to 2.6 cm², 1.9 to 2.5 cm², more preferably in the range of 2.0 to 2.4 cm², and most preferably in the range of 2.2 to 2.4 cm². In a particularly preferred embodiment, the electroactive surface area is about 2.35 cm². In another embodiment, the electroactive surface area of the modified electrode as measured with uric acid is in the range of 0.5 to 2.0 cm², preferably in the range of 1.0 to 1.7 cm², 1.1 to 1.6 cm², more preferably in the range of 1.2 to 1.5 cm², and most preferably in the range of 1.3 to 1.5 cm². In particularly preferred embodiment, the electroactive surface area is about 1.43 cm². In another preferred embodiment, the electroactive surface area of the modified electrode as measured with L-tyrosine is in the range of 0.5 to 1.0 cm² preferably in the range of 0.10 to 0.50 cm², 0.15 to 0.45 cm², more preferably in the range of 0.20 to 0.40 cm², and most preferably in the range of 0.25 to 0.35 cm². In particularly preferred embodiment, the electroactive surface area is about 0.30 cm².

A second aspect of the invention is directed to a method of modifying a graphite pencil electrode comprising:
disolving MTLB in water to form an MTLB solution,
suspending graphene oxide (GO) in the solution, and
reducing MTLB-GO electrochemically on the pencil electrode surface by sweeping electrode potential.

The electrochemical characteristics of the MTLB/GR-modified pencil working electrode are highly dependent on the method of its making. The modification of the pencil graphite base electrode is accomplished by the electrochemical reduction of a composition comprising MTLB and GO at the surface of the graphite pencil electrode. The composition comprising a solution of MTLB in water at a concentration in the range of 0.1 mM to 1.0 mM, preferably in the range of 0.2 mM to 0.8 mM, 0.3 mM 0.7 mM, more preferably in the range of 0.4 mM and 0.6 mM, and most preferably about 0.5 mM; and GO in an amount in the range of 1.0 mg/mL to 4 mg/mL, preferably in the range of 1.5 mg/mL to 3.0 mg/mL, more preferably in the range of 2.0 mg to 2.5 mg/mL, and most preferably about 2.0 mg/mL. The reduction is carried out by scanning the voltage from about −1.4 V to about 0.5 V at scanning rate preferably in the range 0.01 V/s to 0.06 V/s, more preferably 0.02 V/s to 0.05 V/s, and most preferably 0.03 V/s to 0.04 V/s for a number of cycles in the range of 2 to 8, preferably in the range of 3 to 7, more preferably in the range of 4 to 6, and most preferably about 5.

In a preferred embodiment, the charge transfer resistance of the MTLB/GR-modified graphite pencil working electrode is at least 95% less than the charge transfer resistance of the graphite pencil base electrode as the working electrode, and wherein the electroactive area of the graphene-modified graphite pencil working electrode is at least 5 times as that of the graphite pencil base electrode as the working electrode.

The three-dimensional network of MTLB/GR vertical walls network composite covers at least 70%, preferably at least 80%, more preferably at least 90%, and most preferably at least 95% of the surface of the graphene-modified graphite pencil working electrode over an area of 0.2 square millimeters or greater, or 0.5 square millimeters or greater, or 0.8 square millimeters or greater, or 1 square millimeter or greater, or 1.5 square millimeters or greater, or 3 square millimeters or greater.

In one embodiment, the MTLB/GR-modified graphite pencil working electrode may have a distinct interface between the base of the MTLB/GR vertical walls and the surface of the graphite pencil base electrode. In another embodiment, the graphene-modified graphite pencil working electrode has no distinct interface between the base of the MTLB/GR vertical walls and the surface of the graphite pencil base electrode. Rather, the wall base of the MTLB/GR is integrated into or merged with the pencil graphite substrate of the graphite pencil base electrode.

In one embodiment, the pencil graphite substrate of the graphite pencil base electrode is made from beneficiated graphite. In another embodiment, the pencil graphite substrate of the graphite pencil base electrode is made from milled graphite. In still another embodiment, the pencil graphite substrate of the graphite pencil base electrode is made from intercalated graphite, or graphite intercalation compound, non-limiting examples of which include MC₈ (M=K, Rb and Cs), MC₆ (M=Li⁺, Sr²⁺, Ba²⁺, Eu²⁺, Yb³⁺, and Ca²⁺), graphite bisulfate, and halogen-graphite compounds.

The oxidation peak currents of dopamine, uric acid, and L-tyrosine on the surface of the MTLB/GR-modified graphite pencil working electrode of the invention are significantly increased compared to unmodified graphite pencil electrode. The magnitude of the increase varies with the anylate. In some instances, the increase is at least 4 times, preferably at least 5 times, more preferably at least 14 times, even more preferably at least 20 times, and most preferably at least 25 times. In some other instances, the magnitude of the increase at least 30, 40, 50, 60 times, or more.

In the disclosed MTLB/GR-modified graphite pencil electrode system, the counter electrode, along with the working electrode, provides a circuit over which current is measured. The potential of the counter electrode can be adjusted to balance the reaction occurring at the working electrode. The counter electrode can be made of a material that doesn't react with the bulk of the analyte solution and conducts well. The counter electrode of the present disclosure can be fabricated from a conducting or semiconducting material such as platinum, gold, or carbon.

In the disclosed MTLB/GR-modified graphite pencil electrode system, the reference electrode provides a stable and well-known electrode potential, against which the potential of the working electrode is measured. The potential of the reference electrode in the electrochemical instrument of the present disclosure is defined as zero (“0”). A potential of the working electrode that is lower than the reference electrode means the potential is negative, and a potential of the working electrode that is higher than the reference electrode means the potential is positive. The stability of the reference electrode in the disclosed electrode system is maintained by not passing current over it. The counter electrode passes all the current needed to balance the current observed at the working electrode. In one embodiment, the reference electrode is an Ag/AgCl reference electrode. In another embodiment, the reference electrode is a hydrogen electrode. In another embodiment, the reference electrode is a saturated calomel electrode. In another embodiment, the reference electrode is a copper-copper (II) sulfate electrode. In still another embodiment, the reference electrode is a palladium-hydrogen electrode.

The MTLB/GR-modified graphite pencil electrode system of the present disclosure may have more than three electrodes. For example, it may have two distinct and separate working electrodes, at least one of which is the MTLB/GR-modified graphite pencil electrode, and which can be used to scan or hold potentials independently of each other. Both of the electrodes are balanced by a single reference and counter combination for an overall four electrode design.

A third aspect of the disclosure is related to a method of detecting any electroactive analyte using the electrode system which includes the MTLB/GR-modified graphite pencil working electrode described herein, a counter electrode, and a reference electrode. As used herein, the term “electroactive compound” is any organic or inorganic compound that undergoes oxidation/reduction in a solution by electric current such as, but not limited to phenol, catechol, benzoquinone, anthraquinone, vitamin C, dopamine, L-tyrosine, uric acid, NADH/NAD, glucose, fructose, and the like. In a preferred embodiment the electroactive molecule is found in biological fluids such as but not limited to whole blood, plasma, urine, saliva, and sweat.

In a preferred embodiment, the method is a diagnostic method for the simultaneous detection of dopamine, uric acid, and L-tyrosine in solution. The method includes contacting the solution with the MTLB/GR-modified graphite pencil electrode system described herein, and conducting voltammetry, preferably differential pulse voltammetry, preferably cyclic voltammetry, or more preferably square wave voltammetry, to detect and/or determine simultaneously the concentrations of dopamine, uric acid, and L-tyrosine in the solution. The square wave voltammetry is conducted by (a) applying a pulsed potential to the MTLB/GR-modified graphite pencil working electrode while sweeping the potential of the MTLB/GR-modified graphite pencil working electrode from a potential that is less than an oxidation peak potential of dopamine, uric acid, and L-tyrosine in the solution and defined as the adsorption potential positively to a potential that is at least the oxidation peak potential of dopeamine, uric acid, and L-tyrosine in the solution, and (b) recording the amount of a forward pulse current and a reverse pulse current during each square wave cycle.

In other embodiments, the amplitude of the pulsed potential is in the range of 20 to 80 mV, preferably in the range of 30 to 70 mV, more preferably in the range of 40 to 60 mV, and most preferably 45 to 55 mV. In a particularly preferred embodiment, the amplitude of the pulse is about 50 mV.

In other embodiments, the voltage step of the square wave voltammetry is in the range of 2 to 10 mV, preferably in the range of 3 to 8 mV, and more preferably in the range of 3 to 5 mV.

In other embodiments, the pH of the solution is in the range of 4 to 13, preferably from 5 to 10, more preferably from 5 to 8, even more preferably from 5.5 to 7.5, and most preferably about 5.5 to 6.5. In a particularly preferred embodiment, the pH of the solution is about 6.0.

In other embodiments, the frequency of the pulsed potential of the square wave voltammetry is in the range of 10 to 100 Hz, preferably in the range of 20 to 75 Hz, more preferably about 30-60 Hz, even more preferably in the range of 40 to 60 Hz, most preferably in the range of 45 to 55 Hz. In a particularly preferred embodiment, the frequency is about 50 Hz.

In some embodiments, the adsorption potential of the square wave voltammetry is in the range of 0.0 to 0.5 V, preferably in the range of 0.1 to 0.4 V, and more preferably about 0.3 V. In some embodiments, the adsorption time is in the range of 20 to-300 seconds, preferably in the range of about 50 to 250 seconds, more preferably in the range of 100 to 200 seconds, and most preferably in the range of 120-180 seconds. In a particularly preferred embodiment, the adsorption time is about 150 seconds.

A major advantage of the electrode system disclosed herein is that the observed oxidation peaks for dopamine, uric acid, and L-tyrosine are well resolved in the cyclic voltammogram. The resolution of the oxidation peaks in solution allows the simultaneous detection and quantification of each analyte. The observed oxidation peak potential of dopamine in solution ranges from 0.10 V to about 0.23 V, preferably from 0.12 V to about 0.20 V, more preferably from 0.14 V to 0.18, and most preferably in the range of 0.16 to 0.17. The observed oxidation peak potential of uric acid in solution ranges from 0.25 V to 0.40 V, preferably from 0.25 V to about 0.35 V, more preferably from 0.28 V to 0.32 V, and most preferably in the range of 0.29 to 0.31. The observed oxidation peak potential of L-tyrosine in solution ranges from 0.50 V to about 0.80 V, preferably from t 0.55 V to about 0.75 V, more preferably from 0.60 V to about 0.70, and most preferably at about 0.63.

Another advantage of the electrode system of the invention is that it has high sensitivity in detecting and quantifying dopamine, uric acid, and L-tyrosine. It displays a low limit of detection (LOD) for dopamine in the range of 5 to 25 nM, preferably in the range of 10 to 20 nM, more preferably in the range of 12 to 18 nM, and most preferably in the range of 14 to 16 nM. Similarly, the LOD for uric acid is observed in the range of 20 to 35 nM, more preferably in the range of 22 to 33 nM, more preferably in the range of 24 to 30 nM, and most preferably in the range of 26 to 28 nM. For L-tyrosine, the LOD is determined in the range of 400 to 500 nM, preferably in the range of 420 to 480 nM, more preferably in the range of 440 to 470 nM, and most preferably in the range of 455 to 465 nM.

The presence of biomolecules and common ions does not significantly interfere with the detection of dopamine, uric acid, and L-tyrosine in a solution using the disclosed method. The solution may further comprise one or more biomolecules such as phenylalanine, alanine, glucose, fructose, L-methionine, uric acid, and ascorbic acid, and/or one or more common ions such as Na⁺, K⁺, Li⁺, Ni²⁺, SO₄²⁻, and Cl⁻. Because of no or low interference from other molecules, the method can be used to detect dopamine, uric acid, and L-tyrosine in various solutions, comprising at least one selected from whole blood, plasma, serum, saliva, sweat, urine, washes of tissues, extracts of tissues, amniotic fluid, placental fluid, a pharmaceutical composition, and a dietary composition. The pharmaceutical composition may be dopamine and/or L-tyrosine containing pill, capsule, or injection fluid, or may not supposedly contain dopamine and/or L-tyrosine and be tested for L-tyrosine contamination, particularly at trace amounts. The dietary composition may be derived from L-tyrosine rich food sources such as cheese, soybeans, beef, lamb, pork, fish, chicken, nuts, seeds, eggs, dairy, beans, and whole grains. L-tyrosine from non-aqueous pharmaceutical and dietary compositions may be first extracted with water or a suitable pH adjusted aqueous solution well above or below the isoelectric point of L-tyrosine, such as pH 2-4 or 9-11, with the resulting L-tyrosine containing extract being optionally diluted, before the L-tyrosine is detected and quantified by the disclosed MTLB/GR-modified graphite pencil electrode system and method.

To quantify the concentrations of dopamine, uric acid, and L-tyrosine in solution, the method can further comprise plotting the difference in current between the forward pulse current and the reverse pulse current during each square wave cycle, the difference in current represented by I, against the applied potential of the MTLB/GR-modified graphite pencil working electrode, the applied potential represented by E, to obtain a square wave voltammogram, and measuring the magnitudes of peak changes in I (peak heights) in the square wave voltammogram. If there are other substances in the solution that undergo oxidation within the range of the applied potential of the MTLB/GR-modified graphite pencil working electrode during the square wave voltammetry, their oxidation current peaks can be distinguished from the dopamine, uric acid, and L-tyrosine oxidation current peaks in the square wave voltammogram if there is sufficient separation between the oxidation peaks potentials of the other substances and the oxidation peak potentials of dopamine, uric acid, and L-tyrosine in the solution. In some embodiments, the magnitude of the peak changes in I occurring at the dopamine, uric acid, and L-tyrosine oxidation peak potentials in the square wave voltammogram linearly correlates with the concentration of dopamine, uric acid, and L-tyrosine ranging. The observed linear relationship between I and dopamine and uric acid concentrations is observed in the range between 10 to 2000 nM, preferably in the range of range between 20 to 1500 nM, more preferably in the range between 40 and 1200 nM, and most preferably between 50 and 1000 nM. For L-tyrosine, the linear relationship is observed between 0.3 μM to 160 μM, preferably between 0.5 μM to 110 μM, more preferably from 0.6 μM to 50 μM, and most preferably from about 0.7 μM to 30 μM, in the solution.

In some other embodiments, the method of the present disclosure can be used to detect L-tyrosine derivatives, such as L-DOPA, melanin, and phenylpropanoids.

### EXAMPLE 1

### Materials and Methods

Sodium phosphate monobasic and dipotassium hydrogen phosphate were obtained from BDH (U.K). Ascorbic acid, L-methionine, dopamine, uric acid, L-tyrosine, glucose, fructose, sodium, and potassium chloride were purchased from Sigma-Aldrich (U.S.A). Alanine and phenylalanine were acquired from Fluka (U.S.A). All reagents were prepared with double distilled acquired from the Water Still Aquatron A 4000D (England).

Raman and FTIR spectra were obtained on HORIBA Scientific LabRAM HR Evolution and NICOLET 6700 FT-IR spectrometer, respectively. The electrochemical measurements were carried out using Auto Lab (Netherland), consisting of three electrode system. The working electrodes were GPE, GR/GPE, MTLB/GPE or MTLB-GR/GPE, the counter electrode was platinum, and the reference electrode was Ag/AgCl. TESCAN LYRA 3instrument was used for recording of FE-SEM images. The pH and weight measurements were determined by Accumet® XL50 pH meter and GR-2000, respectively.

### EXAMPLE 2

### Electrode Modification Procedure

Pencil electrodes were modified with graphene oxide (GO) or methylene blue (MTLB)-GO. Prior to modification, GO (2 mg/mL) or a mixture of 0.5 mM MTLB and 2 mg/mL GO were dispersed in double distilled water. The MTLB-GO was reduced on the GPE surface by sweeping electrode potential from −1.4 to 0.5 V over five cycles using scan rate 0.03 Vs⁻¹. The modified surface was gently washed by dipping twice in double distilled water before analysis to remove adsorbs MTLB-GO from the surface.

### EXAMPLE 3

### Performance Enhancement of MTBL-GR-GPE Electrode

GPE is a cost-effective electrode sensor, but it has a drawback related to its surface sensitivity similar to other bare electrodes. In the instant invention, the surface sensitivity was improved by direct electrochemical reduction of MTLB-GO composite on the GPE surface. In order to achieve the 3D architecture of a vertical multiwalls network forming concave structures with improved sensitivity, the reaction conditions leading to the formation of the modified electrode were examined.

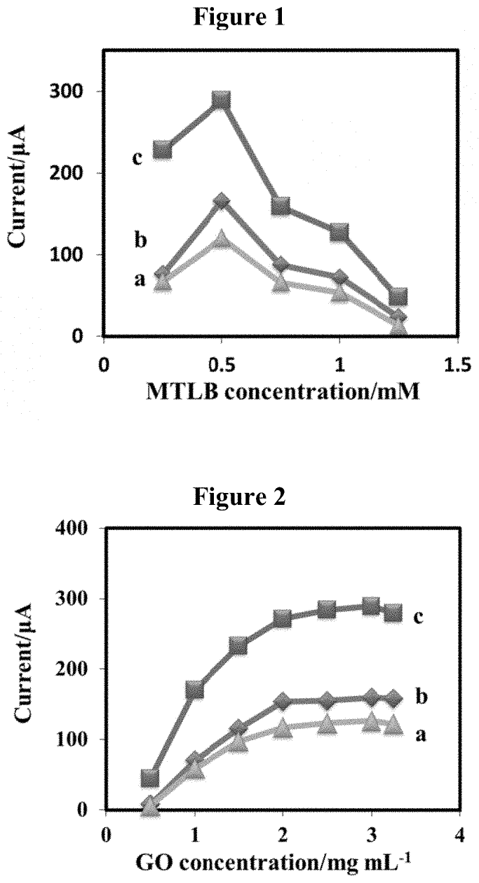

FIGS. 1 and 2 are plots of oxidation peak current response of dopamine, uric acid, and L-tyrosine obtained with electrodes prepared at different concentrations MTLB and GO, respectively. The sensor response was observed for the simultaneous sensing of dopamine, uric acid, and L-tyrosine. A GPE prepared from a solution of MTLB and GO at concentrations of 0.5 mM and the 2 mg/mL, respectively (see FIGS. 1 and 2), provided a strong response.

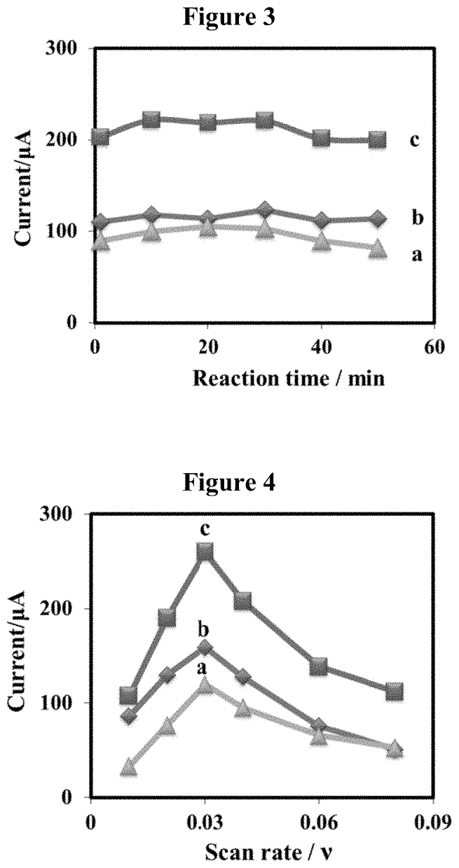

The reaction time of MTLB and GO was analyzed as it may affect the sensor sensitivity and stability of the electrode. FIG. 3 shows a plot of the oxidation peak current response obtained for electrodes prepared with different reaction time. No significant difference in electrode sensitivity is observed for 0.5 mM dopamine, uric acid, and L-tyrosine. The interaction between GO and MTLB is spontaneous which contributes to the development of multiwall network forming a concave 3D architecture of the reduced composite. The fast fabrication process contributes to the low cost of producing a disposable sensor.

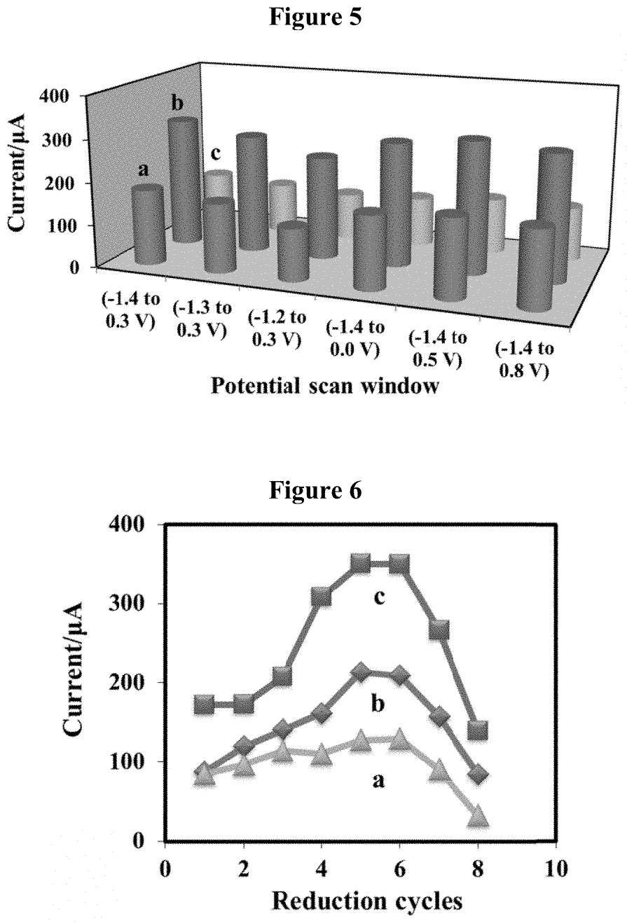

Also, the electrochemical reduction parameters were determined using cyclic voltammetry. The sensitivity of the electrode is improved with scanning rate of 0.03 V/s for the reduction of the MTLB-GO composite, see FIG. 4. The best scan window for the reduction of the mixture was found −1.4 to 0.5 V (see FIG. 5). Finally, the number of scans for the MTLB-GO composite was determined which controls the thickness of the graphene layers and provide the higher sensitivity to the modified GPE. The response was at maximum when reduction cycles were five. Further increase in reduction cycles decreases the sensor sensitivity. The decrease in sensitivity may be due to collapsing of the multiwall network 3D-structure of the reduced graphene oxide composite leading to reorganization of the graphene layers and/or agglomeration of the graphene which negatively affect the sensitivity of the sensor (FIG. 6).

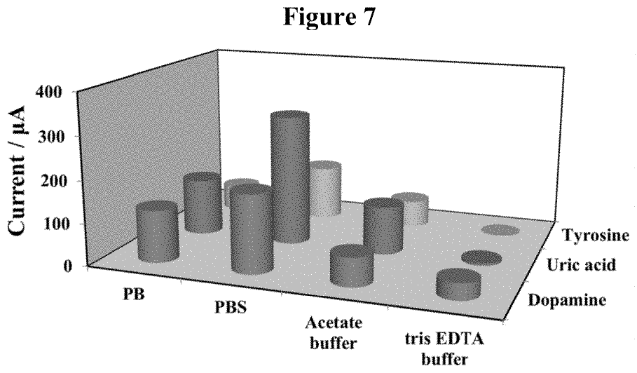

The effect of the buffering medium on the sensor response was examined. The modified GPE response to dopamine, uric acid, and L-tyrosine is examined in phosphate buffer (PB), phosphate buffer saline (PBS), acetate buffer, and tris-EDTA and the results are shown in FIG. 7. The PBS buffer has significantly increased the peak currents of the three analytes. Since PBS contains 0.09 M sodium chloride, the increase peak current is possibly due to the high conductance effect of the medium (FIG. 7).

### EXAMPLE 4

### Characterization of the Modified GPE

### (a) FE-SEM Study:

The surfaces of the bare GPE and modified GPE's were investigated by FE-SEM and Raman spectroscopy. FE-SEM images showed particular changes on the surface of GPE after each modification step. MTLB, GO, and MTLB-GO were reduced on the surface of GPE in a similar fashion under the same conditions. FESEM images of bare and MTLB/GPE surfaces show no layers on the surfaces (FIGS. 8A and 8B). The image of the bare GPE displays irregular surface. Graphene layers were observed on the surface of GR/GPE with few wrinkles. The reduced graphene oxide spread in two-dimension without any 3D extension which is clear from the SEM image (see FIG. 8C). The controlled composition interaction of the methylene blue and the graphene oxide provided a unique surface. The reduction of the composite provided a vertical multiwall network forming concave 3D-structures such as pseudo cup-shapes of the graphene composite on the surface of graphite. The vertical multiwall network forming the concave structures are clear in FIG. 8D. The inner, outer and upper walls of the 3D graphene composite forming the concaves can be clearly seen from the SEM images (see FIG. 8D). The multiwall network of concave structures provides unexpectedly a much larger electroactive surface area than that of the unmodified electrode, and thereby increasing the electroactive surface of the electrode in contact with electrolytes.

### (b) Raman Spectroscopy Study:

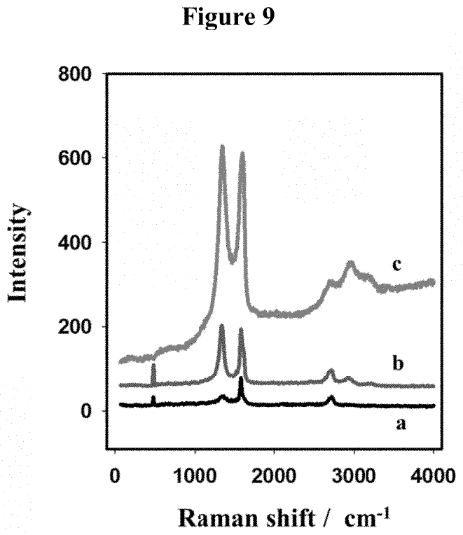

Modified surfaces were examined by Raman spectroscopy study. The Raman spectrum of bare GPE has shown the expected weak D band at 1349 cm⁻¹ and strong G band at 1604 cm⁻¹. The 2D band appeared at 2707 cm⁻¹ (FIG. 9, line a). Although graphene Raman spectra should be similar to graphite, strong D and G bands are observed in GR/GPE spectrum indicating the formation of graphene layers on the GPE surface (see FIG. 9, line b). Similar Raman spectra-like GR/GPE were observed for 3D-MTLB-GR/GPE, but the intensity of D and G band was dramatically enhanced.

### (c) Scanning Rate:

Scan rate effect was studied on GR/GPE and 3D multiwall network structure MTLB-GR composite. The modified surfaces were investigated for dopamine, uric acid, and L-tyrosine. The scan rate was varied from 0.05 to 0.25ν for 0.2 mM dopamine and uric acid using cyclic voltammetry (FIGS. 10 A, B, C, and D). Similarly, it was varied from 0.01 to 0.1ν for 0.5 mM L-tyrosine (FIGS. 10E and F). As the scan rate increased, the current was increased for the same concentration of analytes. However, the current enhancement was much greater for the 3D-MTLB-GR/GPE compared to GR/GPE. The behavior of uric acid unexpectedly changed on the surface of the concave 3D-structure of MTLB-GR composite. The reversible peak of the uric acid became significantly more prominent. This is a clear indication of better performance of the 3D graphene compared 2D graphene due to the availability of more reactive sites which provide fast charge transfer. The electroactive surface area was calculated for the GR/GPE and the MTLB-GR/GPE using equation 1:

Iₚ=2.69×10⁵ ν^{1/2} n^{3/2} C D^{1/2} A, Equation 1

Where Iₚ is the peak current (A), ν is the scan rate (Vs⁻¹), n is the number of electrons, C is the concentration of the analyte (mol L⁻¹), D is the diffusion coefficient (cm²s⁻¹), and A is the electroactive surface area of the electrode (cm²). As pointed out above, the electroactive surface area of the 3D MTLB-GR/GPE was unexpectedly much larger compared to the GR/GPE. The electroactive surface area of dopamine, uric acid and L-tyrosine was increased from 0.141, 0.453 and 0.0445 for GR/GPE to 2.353, 1.43 and 0.299 cm² for 3D-MTLB-GR/GPE (FIGS. 10 E and F). The substantial increase in the electroactive surface area of GPE can only be explained by the concave 3D structures growth of the MTLB-GR composite on the GPE surface.

### EXAMPLE 5

### Impedance and Peak Separation Study

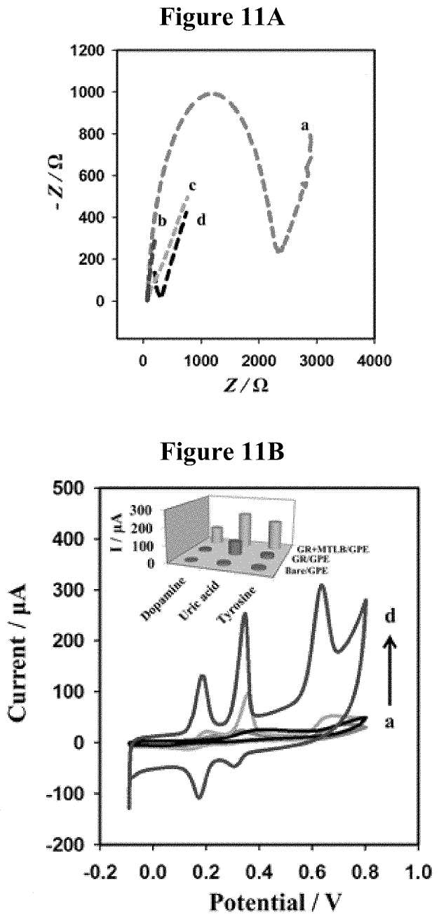

The main purpose of the modification is to overcome the charge transfer resistance of the bare surface of the GPE. The surface resistances were analyzed by the electrochemical impedance spectroscopy (EIS). EIS spectra were scanned from 0.01 to 100 kHz in 0.1 M KCl solution containing five mM K₃Fe(CN)₆/K₄Fe(CN)₆ Nyquist plots displayed two portions. The linear part at lower frequency corresponds to diffusion control process while the semicircle part at higher frequency describes the electron transfer limited process. A large semicircle was observed in the case of bare GPE. The semicircle of the GPE was reduced by GR modification and was almost absent with MTLB-GR/GPE modification (FIG. 11A). The impedance result has shown the multiwall network concave structures of the 3D MTLB-GR composite on the sensor surface facilitate the fast charge transfer for simultaneous sensing of dopamine, uric acid, and L-tyrosine. The CVs were recorded for the 0.2 mM dopamine and uric acid, and 0.5 mM L-tyrosine using bare GPE (FIG. 11Ba), MTLB/GPE (FIG. 11Bb), GR/GPE (FIG. 11Bc), and 3D-MTLB-GR/GPE (FIG. 11 Bd). Although the bare GPE shows peaks for the analytes, the peak currents were very small and were not well resolved. In addition, the peaks were broad which can affect the selectivity of the sensor in the presence of other electroactive species. Similarly, MTLB/GPE was found insensitive. The peak separation and the current were improved by the graphene layer on the GPE. However, the 3D MTLB-GR composite on the GPE has dramatically enhanced the peak current and substantially improved the peak separation of the dopamine, uric acid, and L-tyrosine (FIG. 11B). Surface analysis of the modified GPE revealed the successful formation of the vertical multiwall network structure of MTLB-GR composite with the help of methylene blue controlled composition on the GPE surface. The controlled growth of the vertical multiwall network structure of MTLB-GR substantially improved the sensitivity and selectivity compared to GR/GPE. The possible electrochemical reactions of dopamine and uric acid are shown in scheme 1. The electrochemical reaction of L-tyrosine is reported by Xu et al. [Michrochem. Acta (2005), 151, 47] and also shown in Scheme 1.

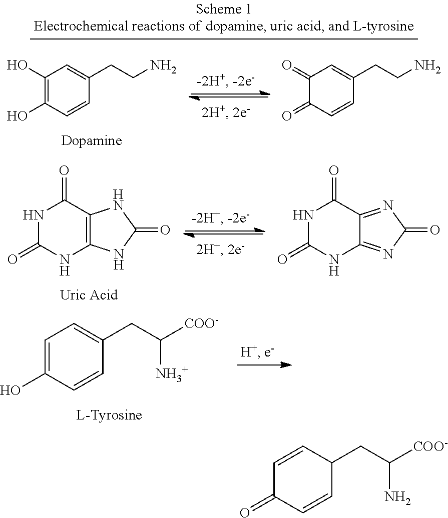

### EXAMPLE 6

### Study of pH Effect

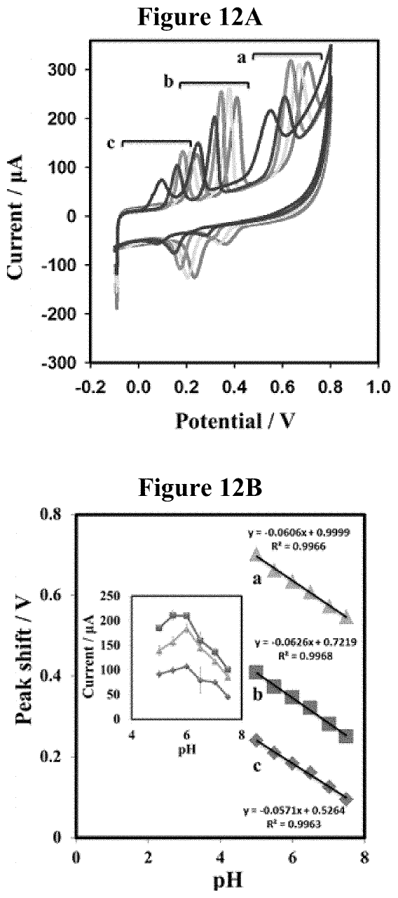

The pH effect was evaluated for 0.2 mM dopamine and uric acid, and 0.4 mM L-tyrosine in 0.1 M PBS buffer medium using cyclic voltammetric scans. The negative peak shifts were observed for dopamine, uric acid, and L-tyrosine as the pH increased from 5.0 to 7.0. The peak shifts of dopamine, uric acid, and L-tyrosine from 241 to 126 mV, 409 to 250 mV, and 701 to 546 mV, respectively. A linear relation was observed between peak shift and the pH change with regression constant (R²) 0.9963, 0.9968 and 0.9966 for dopamine, uric acid, and L-tyrosine, respectively (FIG. 12). The slope For dopamine, uric acid and L-tyrosine, the observed slopes of the lines were −57.1 mV/pH (Eq. 2), −62.6 mV/pH (Eq. 3), and −60.6 mV/pH (Eq. 4), respectively. The pH has shown some effect on the peak current of the analyts. The best response was observed at pH 6.0 which was used for further study.

E vs. Ag/AgCl=526.4−57.1[pH](R²=0.9963) 2

E vs. Ag/AgCl=721.9−62.6[pH](R²=0.9968) 3

E vs. Ag/AgCl=999.9−60.6[pH](R²=0.9966) 4

### EXAMPLE 7

### Sensing Technique

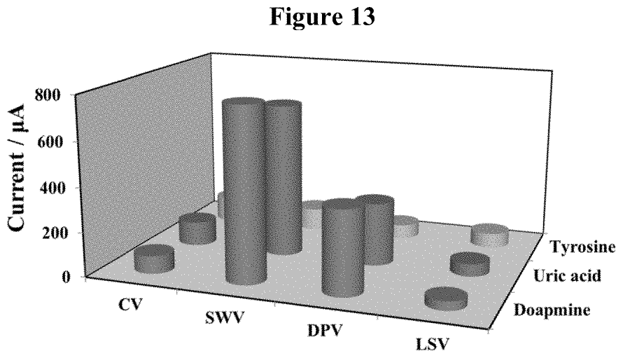

The electrochemical reactions of dopamine, uric acid, and L-tyrosine were examined by various voltammetric techniques and the results are shown in FIG. 13. The best response current was obtained by square wave voltammetry (SWV).

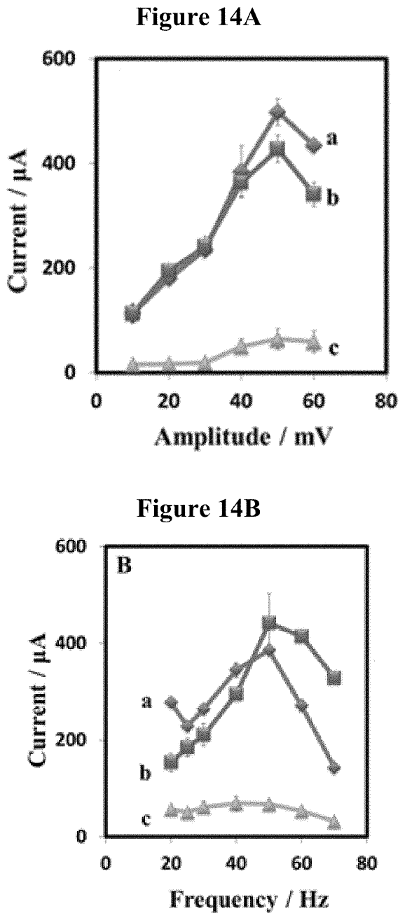

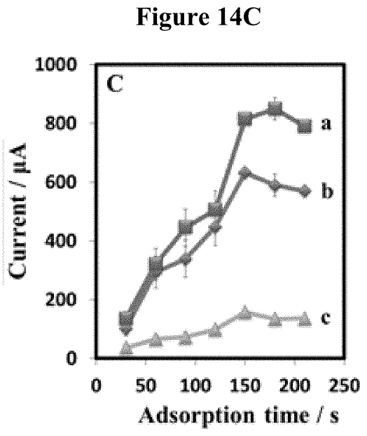

The sensitivity of the sensor was further improved by selecting the parameters of the square wave voltammetry. Initially, the amplitude was measured, and best response was observed at 50 mV (FIG. 14A). Also, the frequency was observed to have a great impact on the peak current, and the best response was observed at 50 Hz (FIG. 14B). In addition, the adsorption time was set for 5 μM dopamine and uric acid, and 40 μM L-tyrosine in 0.1 M PBS. The sensor has shown great affinity to adsorb dopamine and uric acid. The current increased with increasing adsorption time up to 150 s and became almost constant at a longer adsorption time (FIG. 14C). However, the SWV parameters have shown less effect on L-tyrosine compared dopamine and uric acid.

### EXAMPLE 8

### Simultaneous Sensing of Dopamine, Uric Acid, and L-Tyrosine, Limit of Detection

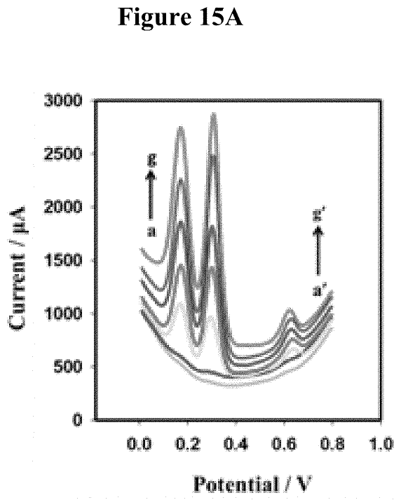

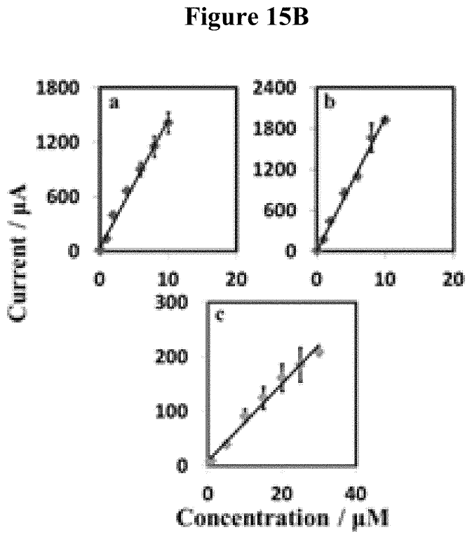

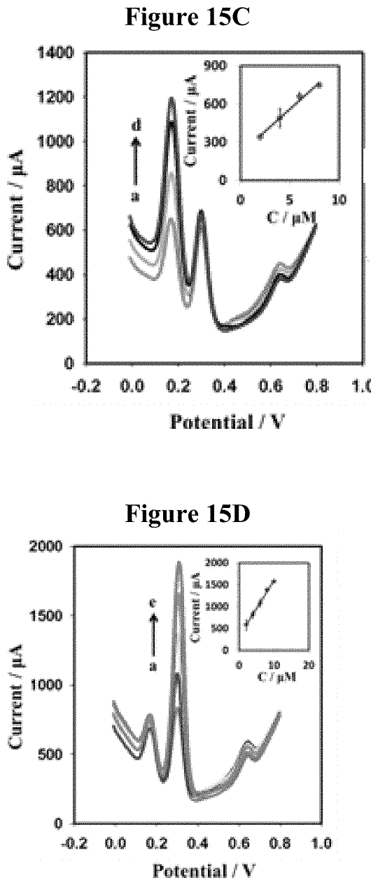

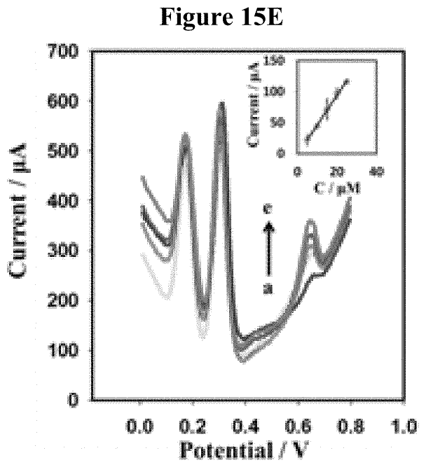

The 3D-MTLB-GR composite sensor was used for the simultaneous sensing of dopamine, uric acid and L-tyrosine in 0.1 M PBS solution. The well-resolved peaks of dopamine, uric acid, and L-tyrosine were observed at 0.167, 0.307 and 0.626 V. The peak separations between dopamine and uric acid, dopamine and L-tyrosine, uric acid and L-tyrosine was found 140 mV, 459 mV, and 319 mV, respectively. The peak separations among the targeted analytes were sufficient for simultaneous sensing. To identify the linear range to develop disposable sensor, various concentrations of the analytes were examined. The sensor was found sensitive to dopamine and uric acid. The linear ranges for dopamine and uric acid were 50 to 10000 nM (FIGS. 15A, Ba, and Bb), whereas that of L-tyrosine was 0.7 to 30 μM (FIGS. 15A and 15Bc). The response of dopamine (FIG. 15C), uric acid (FIG. 15D), and L-tyrosine (FIG. 15E) was considered by varying the concentration of the analytes while keeping the concentration of the other two analytes constant. A linear response of the current and varying analytes concentrations was observed while a small change in current was observed for the constant concentration analytes. The developed disposable sensor is envisioned to be used for individual and/or simultaneous sensing of dopamine, uric acid, and L-tyrosine.

Finally, the sensor was evaluated for reproducibility. Six different 3D-MTLB-GR composite sensors were fabricated under the same set of conditions. The developed sensors were used for the simultaneous analysis of dopamine, uric acid, and L-tyrosine. The RSD values were found 4.02, 5.44 and 6.72% for dopamine, uric acid, and L-tyrosine, respectively.

### EXAMPLE 9

### Distinguish Characteristics of MTLB/GR-3D Composite Sensor Over Other Graphene-Based Sensors

Graphene is continuously being explored in the field of electrochemical sensing of dopamine, uric acid, and L-tyrosine. Limit of quantification of Graphene/Nickel hydroxide/GCE was 120 and 460 nM for dopamine and uric acid, respectively [Nancy et al. “Synergistic electrocatalytic effect of graphene/nickel hydroxide composite for the simultaneous electrochemical determination of ascorbic acid, dopamine and uric acid” Electrochim. Acta. 133 (2014) 233-240. doi:10.1016/j.electacta.2014.04.027]. GO was doped with graphitic carbon nitride nanosheets and detection limits of 96 and 228 nM were achieved for dopamine and uric acid, respectively. The limit of detection found for dopamine and uric acid by Ag NPs/rGO/GCE was 5400 and 8200 nM, respectively [Kaur et al. “Simultaneous and sensitive determination of ascorbic acid, dopamine, uric acid, and tryptophan with silver nanoparticles-decorated reduced graphene oxide modified electrode” Colloids Surfaces B Biointerfaces. 111 (2013) 97-106. doi:10.1016/j.colsurfb.2013.05.023]. In another work, the Pd and Pt NPs were used for fabrication of Pd3Pt1/PDDA-RGO/GCE and limits of detection of dopamine and uric acid were observed of 40 and 100 nM [Yan et al. “Simultaneous electrochemical detection of ascorbic acid, dopamine and uric acid based on graphene anchored with Pd—Pt nanoparticles” Colloids Surfaces B Biointerfaces. 111 (2013) 392-397. doi:10.1016/j.colsurfb.2013.06.030]. Also, methylene blue was used for sensing of various electroactive molecules. PMB-GR on CILE was used for sensing of dopamine [Sun et al. “Poly(methylene blue) functionalized graphene modified carbon ionic liquid electrode for the electrochemical detection of dopamine” Anal. Chim. Acta. 751 (2012) 59-65. doi:10.1016/j.aca.2012.09.006]. Han et al. [“Synthesis of graphene/methylene blue/gold nanoparticles composites based on simultaneous green reduction, in situ growth and self-catalysis” J. Mater. Sci. 49 (2014) 4796-4806. doi:10.1007/s10853-014-8179-2.] cast rGO/MB/AuNPs/GCE nanocomposite on a glassy carbon electrode and used it for the simultaneous sensing of ascorbic acid, dopamine, and uric acid. The detection limit for dopamine and uric acid was found 150 and 250 nM, respectively. The peak separation between dopamine and uric acid was 132 mV.

The present disclosure describes the development of a 3D-MTLB-GR composite modified PGE with the controlled composition of methylene blue. As a result, a vertical multiwall network forming concave structures of MTLB-GR composite grow on the surface of the GPE. The morphology of the MTLB-GR composite was found highly sensitive and selective due to the availability of more exposed active surface area. The combination of fast fabrication time and low cost of making the modified electrode assists in achieving the ultimate goal of developing disposable sensor graphite pencil electrode. The limit of detection for dopamine and uric acid were 15 and 27 nM, respectively. The peak separation between dopamine and uric acid was found 140 mV which more than rGO/MB/AuNPs/GCE and many other graphene-based sensors. Moreover, no precious metals such as Au or Pt NPs were used to achieve high sensitivity. Due to short fabrication time, it can be used as a single-use electrode. Comparison of the vertical multiwall network forming concave 3D-structures of MTLB/GR composite sensor with other graphene based sensor described in Table 1.

TABLE 1
The comparison of the MTLB/GR 3D composite sensor with reported graphene-based sensors

| | Modified | | Sensing | Sensing | LOQ | LOD | | |
|---|---|---|---|---|---|---|---|---|
| Sr# | electrode | Analyte | technique | medium | (nM) | (nM) | Application | Ref. |
| 1 | H-GO/GCE | DA | DPV | B-R/pH 6.0 | 500, | — | Urine & | (a) |
| | | UA | | | 500 | | serum | |
| | | | | | | | samples | |
| 2 | pCu₂O NS-rGO/GCE | DA | DPV | 0.1M | 50, | 15 | — | (b) |
| | | UA | | PB/pH 7.0 | 1000 | 112 | | |
| 3 | GF@NiCo₂O₄ | DA | DPV | 0.1M | 1000, | 100 | Urine & | (c) |
| | | UA | | PBS/pH 7.0 | 10000 | 200 | serum | |
| | | | | | | | samples | |
| 4 | RGO-ZnO/GCE | DA | DPV | 0.1M | 3000, | 1080 | Urine & | (d) |
| | | UA | | PB/pH 6.0 | 1000 | 330 | plasma | |
| | | | | | | | samples | |
| 5 | GR/Au/GR/Au/GPE | DA | SWV | 0.1M | 100, | 24 | Urine | (e) |
| | | UA | | PBS/pH 6.0 | 90 | 29 | sample | |
| 6 | MoS2/rGO/GCE | DA | DPV | 0.1M | 5000, | 50 | Serum | (0 |
| | | UA | | PB/pH 7.0 | 25000 | 460 | sample | |
| 7 | Porous graphene/GCE | DA | DPV | 0.1M | 200, | 200 | — | (g) |
| | | UA | | PB/pH 6.8 | 1000 | 1000 | | |
| 8 | AuNCs/AGR/ | DA | SWVs | 0.1M | 1000, | 80 | Urine | (h) |
| | MWCNT/GCE | UA | | PB/pH 7.0 | 50000 | 100 | sample | |
| 9 | rGO/MB/AuNPs/GCE | DA | DPV | PBS/7.4 | | 150 | | (i) |
| | | UA | | | | 250 | | |
| 10 | Trp-GR/GC | DA | DPV | 0.1M | 500, | 290 | Injection, | (j) |
| | | UA | | PB/pH 7.0 | 10000 | 1240 | Urine & | |
| | | | | | | | serum | |
| | | | | | | | samples | |
| 11 | RGO-PAMAM- | DA | DPV | 0.1M | 10000, | 3330 | — | (k) |
| | MWCNT-AuNP/GCE | UA | | PB/pH 4.0 | 10000 | 330 | | |
| 12 | 3D-MTLB-GR/GPE | DA | SWV | 0.1M | 50, | 15 | Urine | This |
| | | UA | | PBS/pH | 50 | 27 | samples | work |

(a) Zou et al. “A novel electrochemical biosensor based on hemin functionalized graphene oxide sheets for simultaneous determination of ascorbic acid, dopamine and uric acid, Sensors Actuators B Chem. 207 (2015) 535-541. doi:10.1016/j.snb.2014.10.121
(b) Mei et al. “A glassy carbon electrode modified with porous Cu2O nanospheres on reduced graphene oxide support for simultaneous sensing of uric acid and dopamine with high selectivity over ascorbic acid” Microchim. Acta. 183 (2016) 2039-2046. doi:10.1007/s00604-016-1845-0.
(c) Cai et al. “Sensors and Actuators B: Chemical Controlled functionalization of flexible graphene fibers for the simultaneous determination of ascorbic acid, dopamine and uric acid” Sensors Actuators B Chem. 224 (2016) 225-232.
(d) Zhang et al. “One-pot facile fabrication of graphene-zinc oxide composite and its enhanced sensitivity for simultaneous electrochemical detection of ascorbic acid, dopamine and uric acid” Sensors Actuators B Chem. 227 (2016) 488-496. doi:10.1016/j.snb.2015.12.073.
(e) Baig et al. “A cost-effective disposable graphene-modified electrode decorated with alternating layers of Au NPs for the simultaneous detection of dopamine and uric acid in human urine” RSC Adv. 6 (2016) 80756-80765. doi:10.1039/C6RA10055D.
(f) Xing et al. “A glassy carbon electrode modified with a nanocomposite consisting of MoS2 and reduced graphene oxide for electrochemical simultaneous determination of ascorbic acid, dopamine, and uric acid” Microchim. Acta. 183 (2016) 257-263. doi:10.1007/s00604-015-1648-8.
(g) Wang et al. “Three-dimensional porous graphene for simultaneous detection of dopamine and uric acid in the presence of ascorbic acid” J. Electroanal. Chem. 782 (2016) 76-83. doi:10.1016/j.jelechem.2016.09.050
(h) Abdelwahab et al. “Simultaneous determination of ascorbic acid, dopamine, uric acid and folic acid based on activated graphene/MWCNT nanocomposite loaded Au nanoclusters” Sensors Actuators B Chem. 221 (2015) 659-665. doi:10.1016/j.snb.2015.07.016.
(i) Han et al. “Synthesis of graphene/methylene blue/gold nanoparticles composites based on simultaneous green reduction, in situ growth and self-catalysi” J. Mater. Sci. 49 (2014) 4796-4806. doi:10.1007/s10853-014-8179-2.
(j) Lian et al. “Simultaneous determination of ascorbic acid, dopamine and uric acid based on tryptophan functionalized graphene” Anal. Chim. Acta. 823 (2014) 32-39. doi:10.1016/j.aca.2014.03.032.
(k) Wang et al. “Simultaneous determination of dopamine, ascorbic acid and uric acid using a multi-walled carbon nanotube and reduced graphene oxide hybrid functionalized by PAMAM and Au nanoparticles” Anal. Methods. 7 (2015) 1471-1477. doi:10.1039/C4AY02086C

### EXAMPLE 10

### Application and Interferences Study

The sensor capability for simultaneous sensing of dopamine, uric acid, and L-tyrosine was carried out in the presence of various potential interfering compounds. The interferences were studied for 4 μM dopamine and uric acid, and 20 μM L-tyrosine. Ascorbic acid is considered a primary interfering agent in the simultaneous sensing of dopamine and uric acid. The dopamine, uric acid, and L-tyrosine were measured in the presence of a high concentration of ascorbic acid (500 μM), and current variation was observed 2.78, 1.30 and 7.07%, respectively. Also, the response of dopamine was observed in the presence of 50 μM other potential interfering compounds such as L-alanine, L-phenylalanine, L-methionine, glucose, and fructose. The current variation was observed in the ranges of 0.8-3.5%, 1.9-12.5%, 2.8-11% for dopamine, uric acid, and L-tyrosine, respectively. A small variation in current of the targeted analytes observed in the presence of interfering compounds.

The 3D-MTLB-GR/GPE was utilized for simultaneous sensing of dopamine, uric acid, and L-tyrosine in human urine sample comprising large amount of uric acid. The urine sample was diluted to bring the concentration of uric acid into the linear range, and a sharp peak of uric acid was observed in a sample without the addition of any interfering compound. The urine sample was not treated chemically before analysis. Standard addition method was applied for the determination of analyte concentrations in the human urine. The urine sample was spiked with 2, 4, 6 μM dopamine and uric acid. Similarly, 10, 15 and 20 μM of L-tyrosine spiked in the urine sample to find out the accuracy of the sensor. The measured concentration of dopamine, uric acid and L-tyrosine were found in the range of 91 to 107% of the actual concentration (Table 2). Satisfactory concentration measurements in biological samples indicated that the developed sensor can be used for quantitative analysis of dopamine, uric acid, and L-tyrosine for diagnostic purposes.

TABLE 2
Determination of dopamine, uric acid and L-tyrosine by
3D-MTLB-GR/GPE in the human urine sample.

| Sr # | Found | Spiked, μM | Measured, μM | % recovery |
|---|---|---|---|---|
| Dopamine | | | | |
| 1 | 0 | 2 | 2.139 | 106.96 |
| 2 | 0 | 4 | 3.656 | 91.38 |
| 3 | 0 | 6 | 6.309 | 105.15 |
| Uric acid | | | | |
| 1 | 3 | 2 | 2.063 | 103.16 |
| 2 | 3 | 4 | 3.787 | 94.68 |
| 3 | 3 | 6 | 6.553 | 109.31 |
| L-tyrosine | | | | |
| 1 | 0 | 10 | 9.197 | 91.97 |
| 2 | 0 | 15 | 13.925 | 92.83 |
| 4 | 0 | 20 | 20.159 | 100.79 |

The 3D architecture of MTLB-GR composite was developed by using a new approach on the surface of cost-effective graphite pencil electrode. It was achieved by controlled interaction and reduction of a composition of MTLB-GO which result in a 3D vertical multiwall network forming concave structures. The 3D structure of the MTLB-GR composite provided a large surface area for the electrochemical reaction resulted from the accessibility of both side of the multiwall network to the analytes. The electroactive surface area of the 3D MTLB-GR composite was improved from 0.141, 0.453 and 0.0445 (GR/GPE) to 2.353, 1.43 and 0.299 cm² for dopamine, uric acid, and L-tyrosine, respectively. The 3D-MTLB-GR/GPE provided enhanced charge transfer due to the presence of large reactive sites for the electrochemical reaction. The 3D MTLB-GR composite sensor has shown low limit of detection of 15, 27, and 247 nM for dopamine, uric acid, and L-tyrosine, respectively. Fully resolved peaks of dopamine and uric acid were observed. The fabricated 3D MTLB-GR composite sensor has displayed the capability to cope with potential interferences. The real sample applications have shown satisfactory recoveries of dopamine, uric acid and L-tyrosine in human urine in the range of 91 to 107%. Due to low cost, facile fabrication and low limit of detection, the 3D MTLB-GR composite modified graphite pencil electrode can be proved a valuable tool for simultaneous sensing of small molecules which is not limited to dopamine, uric acid, and 1-tyrosine.

1: A graphene-modified graphite pencil electrode system, comprising:
a graphene-modified graphite pencil working electrode comprising a graphite pencil base electrode modified with a three-dimensional network of vertical walls of methylene blue (MTLB)/graphene composite forming concave structures on the surface of the graphite pencil base electrode,
a counter electrode, and
a reference electrode.

2: The graphene-modified graphite pencil electrode system of claim 1, wherein the graphene-modified pencil working electrode has an electroactive surface areas determined for dopamine, uric acid, and L-tyrosine of about 2.35 cm², 1.43 cm², and 0.30 cm², respectively.

3: The graphene-modified graphite pencil electrode system of claim 1, wherein the graphene-modified pencil working electrode is obtained by electrochemical reduction of a composition comprising MTLB and graphene oxide (GO) at the surface of a graphite pencil electrode by scanning from −1.4 to 0.5 V at scan rate in the range of 0.02 to 0.04 V/s for 4 to 6 cycles.

4: The graphene-modified graphite pencil electrode system of claim 3, wherein the composition comprises MTLB at a concentration in the range of 0.4 to 0.6 mM and GO at a concentration of at least 2 mg/mL.

5: The graphene-modified graphite pencil electrode system of claim 1, wherein the charge transfer resistance of the graphene-modified graphite pencil working electrode is at least 95% less than the charge transfer resistance of an unmodified graphite pencil base electrode as the working electrode, and wherein the electroactive area of the graphene-modified graphite pencil working electrode is at least 5 times as that of an unmodified graphite pencil base electrode as the working electrode.

6: A method of modifying graphite pencil electrode comprising:
disolving methylene blue (MTLB) in water at a concentration in the range of 0.4 to 0.6 mM to form an MTLB solution,
suspending graphene oxide (GO) in the solution in an amount in the range of 1.5 to 3.0 mg/mL, and
reducing MTLB-GO on the pencil electrode surface by sweeping electrode potential from about −1.4 to about 0.5 V over 4 to 7 cycles at scanning rate in the range of 0.02 to 0.04 V/s.

7: A method of detecting dopamine, uric acid, L-tyrosine, or combination thereof simultaneously in a solution, comprising:
contacting the solution with the graphene-modified graphite pencil electrode system of claim 1, and
conducting square wave voltammetry to determine one or more concentrations of dopamine, uric acid, and L-tyrosine in the solution, wherein the conducting square wave voltammetry comprises:
(a) applying a pulsed potential to the graphene-modified graphite pencil working electrode while sweeping the potential of the graphene-modified graphite pencil working electrode from a potential that is less than an oxidation peak potential of dopamine, uric acid, and L-tyrosine in the solution and defined as the adsorption potential positively to a potential that is at least the oxidation peak potential of dopamine, uric acid, and L-tyrosine in the solution, and
(b) recording the amount of a forward pulse current and a reverse pulse current during each square wave cycle.

8: The method of claim 7, wherein the amplitude of the pulsed potential is in the range 10 to 100 mV.

9: The method of claim 7, wherein the voltage step of the square wave voltammetry is in the range of about 2 to 10 mV.

10: The method of claim 7, wherein the pH of the solution ranges from about

5. 0 to 7.0.

11: The method of claim 7, wherein the frequency of the pulsed potential is in the range of about 25 to 75 Hz.

12: The method of claim 7, wherein the oxidation peak potential of dopamine in the range of 0.10 to 0.20 V, uric acid in the range 0.25 to 0.35 V, and L-tyrosine in the range of 0.5 V to 0.7 V in the solution.

13: The method of claim 7, wherein the sweeping the potential of the graphene-modified graphite pencil working electrode from the adsorption potential is to adsorb dopamine, uric acid, and L-tyrosine in the solution to the surface of the graphene-modified graphite pencil working electrode.

14: The method of claim 13, wherein the adsorption time is in the range of 100 to 200 seconds.

15: The method of claim 7, wherein the lowest detectable dopamine, uric acid, and L-tyrosine concentrations in the solution are about 15, 27, and 247, respectively.

16: The method of claim 7, wherein the solution further comprises one or more selected from the group consisting of ascorbic acid, L-phenylalanine, L-alanine, glucose, fructose, L-methionine, uric acid, ascorbic acid, Na⁺, K⁺, Li⁺, Ni²⁺, SO₄²⁻, and Cl⁻.

17: The method of claim 7, wherein the solution comprises at least one selected from the group consisting of whole blood, plasma, serum, saliva, sweat, urine, washes of tissues, extracts of tissues, amniotic fluid, placental fluid, a pharmaceutical composition, and a dietary composition.

18: The method of claim 7, further comprising plotting the difference in current between the forward pulse current and the reverse pulse current during each square wave cycle, the difference in current represented by I, against the applied potential of the graphene-modified graphite pencil working electrode, the applied potential represented by E, to obtain a square wave voltammogram, and measuring the magnitudes of peak changes in I in the square wave voltammogram.

19: The method of claim 18, wherein the magnitude of the peak change in I occurring at the dopamine, uric acid, and L-tyrosine oxidation peaks potential in the square wave voltammogram linearly correlates with the concentration of dopamine and uric acid in the range of 50 to 1000 nM, and L-tyrosine is in the range from about 0.7 μM to 30 μM in the solution.

20: A method of simultaneous determination of dopamine, uric acid, and L-tyrosine concentrations in a solution, comprising:
contacting the solution with the graphene-modified graphite pencil electrode system of claim 1, and
conducting square wave voltammetry to determine dopamine, uric acid, and L-tyrosine concentrations in the solution, wherein the conducting square wave voltammetry comprises:
(a) applying a pulsed potential to the graphene-modified graphite pencil working electrode while sweeping the potential of the graphene-modified graphite pencil working electrode from a potential that is less than an oxidation peak potential of uric acid in the solution and defined as the adsorption potential positively to a potential that is at least the oxidation peak potential of L-tyrosine in the solution, and
(b) recording the amount of a forward pulse current and a reverse pulse current during each square wave cycle,
wherein the square wave voltammetry includes conditions in which: the frequency is in the range of 40 to 60 Hz; the amplitude is is in the range of 20 to 80 mV; the voltage step is in the range of 2 to 10 mV; the adsorption potential is in the range of 0.0 to 0.4 V; the adsorption time is in the range 100 to 200 seconds; and the pH value is in the range of 5.0 to 7.0.
